(19) United States
(12) Patent Application Publication
(10) Pub. No.: US 2026/0207143 A1
(43) Pub. Date: Jul. 23, 2026

(54) BIOLOGICAL MONITORING DEVICE

(71) Applicant: SEIKO GROUP CORPORATION, Tokyo (JP)

(72) Inventors: Ryosuke ISOGAI, Matsudo-shi (JP); Yoshifumi YOSHIDA, Matsudo-shi (JP)

(21) Appl. No.: 19/140,239

(22) PCT Filed: Dec. 6, 2023
(86) PCT No.: PCT/JP2023/043643

§ 371 Date: Jun. 17, 2025

(30) Foreign Application Priority Data
Jan. 12, 2023 (JP) 2023--003002

(51) Int. Cl. A61B 5/00; A61B 5/07

(52) U.S. Cl. CPC A61B 5/6882; A61B 5/076; A61B 5/688; A61B 5/6884; A61B 2560/0276

(57) ABSTRACT

A biological monitoring device includes a sensor unit substrate on which one or more sensors are mounted and a control unit substrate on which a power source, a control unit and a storage unit are mounted, wherein the sensor unit substrate has a sensor-mounted region in which the sensor is mounted and an extraction region having a power source line and a signal line between it and the control unit substrate, and the sensor-mounted region is implanted in a living body.

### RELATED APPLICATIONS

This application is a 371 application of PCT/JP 2023/043643 having an international filing date of Dec. 6, 2023, which claims priority to JP 2023-003002 filed Jan. 12, 2023, the enter content of each of which is incorporated herein by reference.

### TECHNICAL FIELD

The present invention relates to a biological monitoring device.

### BACKGROUND ART

Conventionally, medical sensors that are implanted in the human body to be used are known (for example, refer to Patent Document 1). Patent Document 1 describes a medical sensor including an electronic device including a sensor with an accelerometer, a bidirectional wireless communication system that is electronically connected to the electronic device in order to transmit an output signal from the sensor to an external device and receive a command from an external controller to the electronic device, and a wireless power source system for supplying power to the electronic device.

### CITATION LIST

### Patent Document

Patent Document 1: Published Japanese Translation No. 2021-513895 of the PCT International Publication

### SUMMARY OF INVENTION

### Technical Problem

However, in the medical sensor described in Patent Document 1, in order to wirelessly supply power to the medical sensor implanted in the body, an antenna and a circuit for wirelessly supplying power are necessary, but the size of the medical sensor increases due to the antenna and the circuit. Accordingly, there is a problem of a heavy burden on the human body in which the medical sensor is implanted. In addition, a power supply device that wirelessly supplies power to the medical sensor should be installed near the human body. In addition, when the medical sensor is applied to small animals, if the size of the medical sensor becomes large, the small animals may not be able to perform their natural activities, and biological information obtained by the medical sensor may no longer be effective for biological analysis of small animals.

The present invention has been made in view of such circumstances, and an object of the present invention is to reduce the burden on the living body in which a sensor is implanted.

### Solution to Problem

One aspect of the present invention provides a biological monitoring device, including: a sensor unit substrate on which one or more sensors are mounted; and a control unit substrate on which a power source, a control unit and a storage unit are mounted, wherein the sensor unit substrate has a sensor-mounted region in which the sensor is mounted and an extraction region having a power source line and a signal line between it and the control unit substrate, and the sensor-mounted region is implanted in a living body.

One aspect of the present invention is the above biological monitoring device, wherein the sensor unit substrate has a displacement prevention unit that prevents the position of the sensor unit substrate in the sensor-mounted region from being shifted.

One aspect of the present invention is the above biological monitoring device, wherein the displacement prevention unit has a protrusion.

One aspect of the present invention is the above biological monitoring device, wherein the displacement prevention unit has a hole.

One aspect of the present invention is the above biological monitoring device, wherein the displacement prevention unit has a protrusion and a hole.

One aspect of the present invention is the above biological monitoring device, wherein the displacement prevention unit is a part where at least a part of the surface of the sensor unit substrate has been surface-treated with a chemical substance that promotes adhesion to biological tissue.

One aspect of the present invention is the above biological monitoring device, wherein the displacement prevention unit is a fine unevenness provided on at least a part of the surface of the sensor unit substrate.

One aspect of the present invention is the above biological monitoring device, wherein the displacement prevention unit is made of a bioabsorbable material.

One aspect of the present invention is the above biological monitoring device, wherein the displacement prevention unit is positioned on the outer periphery of the sensor unit substrate.

One aspect of the present invention is the above biological monitoring device, wherein the displacement prevention unit is disposed substantially perpendicular to an interface between the epidermis and the subcutaneous layer.

One aspect of the present invention is the above biological monitoring device, wherein the sensor unit substrate has a plurality of displacement prevention units.

One aspect of the present invention is the above biological monitoring device, wherein the sensor unit substrate has a plurality of displacement prevention units at positions that do not overlap when the sensor unit substrate is viewed from above.

One aspect of the present invention is the above biological monitoring device, wherein the sensor-mounted region has a fall-off prevention structure on the side of the extraction region to prevent the sensor-mounted region from falling off of the living body.

One aspect of the present invention is the above biological monitoring device, wherein the sensor-mounted region has a rounded shape on the side opposite to the extraction region.

One aspect of the present invention is the above biological monitoring device, wherein an anti-adhesion agent is applied to the periphery of the sensor in the sensor-mounted region.

One aspect of the present invention is the above biological monitoring device, wherein, in the sensor unit substrate, the sensor is mounted on the surface that is inside the body when the sensor is implanted in the living body between both surfaces of the sensor-mounted region.

One aspect of the present invention is the above biological monitoring device, wherein either a temperature sensor or a pulse wave sensor, or both a temperature sensor and a pulse wave sensor are mounted as the sensor on the surface that is inside the body.

One aspect of the present invention is the above biological monitoring device, wherein, in the sensor unit substrate, ends of the sensor-mounted region are chamfered.

One aspect of the present invention is the above biological monitoring device, wherein, on the control unit substrate, a wireless communication unit is additionally mounted, and the control unit causes the wireless communication unit to wirelessly transmit a sensor output signal stored in the storage unit to an external device.

One aspect of the present invention is the above biological monitoring device, further including a clock unit, wherein the storage unit stores the sensor output signal in association with a recording time, which is a time measured by the clock unit, the control unit causes the wireless communication unit to wirelessly transmit a predetermined signal to the external device at a predetermined transmission period based on the time measured by the clock unit, the control unit causes the wireless communication unit to wirelessly transmit the sensor output signal and the recording time associated with the sensor output signal to the external device, and the external device corrects time series data of the sensor output signal and the recording time wirelessly received by the external device based on a reception time of the predetermined signal wirelessly received by the external device.

One aspect of the present invention is the above biological monitoring device, wherein the control unit causes the wireless communication unit to wirelessly transmit a signal indicating an operation status of the biological monitoring device to the external device.

One aspect of the present invention is the above biological monitoring device, wherein the control unit notifies a user of whether the installation position of the sensor is normal.

One aspect of the present invention is the above biological monitoring device, wherein the sensor-mounted region and the control unit substrate are non-flexible, and the extraction region is flexible.

One aspect of the present invention is the above biological monitoring device, wherein, in the sensor unit substrate, the extraction region is disposed across the inside of the living body and the outside of the living body, and the control unit substrate is disposed outside the living body.

One aspect of the present invention is the above biological monitoring device, wherein the extraction region has a constricted shape.

One aspect of the present invention is the above biological monitoring device, wherein the extraction region has a plurality of constricted shapes in a direction in which the extraction region extends.

One aspect of the present invention is the above biological monitoring device, wherein the extraction region has a constricted shape having a width smaller than both ends on the side of the sensor-mounted region and on the side of the control unit substrate.

One aspect of the present invention is the above biological monitoring device, wherein the extraction region has one or more curved parts.

One aspect of the present invention is the above biological monitoring device, wherein the extraction region has a meander shape formed by a plurality of curved parts.

One aspect of the present invention is the above biological monitoring device, wherein the extraction region has a wedge shape.

One aspect of the present invention is the above biological monitoring device, wherein the sensor unit substrate has a pressure applying mechanism for pressing the sensor-mounted region against the inside of the living body.

One aspect of the present invention is the above biological monitoring device, wherein at least one of the sensor unit substrate and the control unit substrate has a connector region that fits into a connector.

One aspect of the present invention is the above biological monitoring device, wherein the connector is disposed on the side of the control unit substrate of the sensor unit substrate that is disposed outside the living body.

One aspect of the present invention is the above biological monitoring device, wherein the control unit substrate has the connector region that fits into a control unit-side connector as the connector, the control unit-side connector is also able to be used for connection to the external device, and the control unit or the storage unit is operable from the external device connected to the control unit substrate via the control unit-side connector.

One aspect of the present invention is the above biological monitoring device, wherein the sensor unit substrate has the connector region that fits into a sensor-side connector as the connector, the sensor-side connector is also able to be used for connection to the external device, and the sensor is operable from the external device connected to the sensor unit substrate via the sensor-side connector.

One aspect of the present invention provides a biological monitoring device, including: a sensor unit substrate on which one or more sensors are mounted; and a control unit substrate on which a power source, a control unit and a storage unit are mounted, wherein the sensor unit substrate has a sensor-mounted region in which the sensor is mounted, an extraction region having a power source line and a signal line between it and the control unit substrate, and a connector region in which a sensor-side connector that fits into a control unit-side connector of the control unit substrate is mounted, the sensor unit substrate and the control unit substrate are detachable using the sensor-side connector and the control unit-side connector, in the sensor unit substrate, the sensor-mounted region is implanted in a living body, in the control unit substrate, the control unit-side connector is used by being connected to the sensor-side connector, the control unit-side connector and the sensor-side connector connect the power source line and the signal line between the side of the control unit substrate and the side of the sensor unit substrate, the sensor operates by receiving power from the power source, and the storage unit stores a sensor output signal output from the sensor.

### Advantageous Effects of Invention

According to the present invention, an effect of reducing the burden on the living body in which the sensor is implanted is obtained.

### BRIEF DESCRIPTION OF DRAWINGS

FIG. 1 is a block diagram showing an example of a configuration of a biological monitoring system according to one embodiment.

FIG. 2 is a plan view showing an example of a mounting configuration of a sensor unit substrate according to one embodiment.

FIG. 3 is a cross-sectional view showing a example of a substrate configuration of a sensor unit substrate according to one embodiment.

FIG. 4 includes a plan view and a cross-sectional view showing a example of a substrate configuration of metal wires in an extraction region according to one embodiment.

FIG. 5 includes a plan view and a cross-sectional view showing a example of a substrate configuration of metal wires in an extraction region according to a comparative example.

FIG. 6 includes a plan view and a cross-sectional view showing a example of a substrate configuration of metal wires in an extraction region according to a comparative example.

FIG. 7 is an illustrative diagram showing an example of usage of a biological monitoring device according to one embodiment.

FIGS. 8A, 8B, 8C and 8D are diagrams showing an example of processing a sensor output signal according to one embodiment.

FIG. 9 is a plan view showing an example of a mounting configuration of a sensor unit substrate according to one embodiment.

FIG. 10 is a plan view showing an example of a mounting configuration of a sensor unit substrate according to one embodiment.

FIG. 11 is a plan view showing an example of a mounting configuration of a sensor unit substrate according to one embodiment.

FIG. 12 includes a plan view and a cross-sectional view showing an example of a mounting configuration of a displacement prevention unit according to one embodiment.

FIG. 13 includes a plan view and a cross-sectional view showing an example of a mounting configuration of a displacement prevention unit according to one embodiment.

FIG. 14 includes a plan view and a cross-sectional view showing an example of a mounting configuration of a displacement prevention unit according to one embodiment.

FIG. 15 is a diagram showing an example of a method of inserting a sensor-mounted region into a living body according to one embodiment.

FIG. 16 is an illustrative diagram showing an example of usage of a biological monitoring device having an fall-off prevention structure according to one embodiment.

FIG. 17 is an illustrative diagram showing an example of usage of a biological monitoring device having a pressure applying mechanism according to one embodiment.

### DESCRIPTION OF EMBODIMENTS

Hereinafter, an embodiment of the present invention will be described with reference to the drawings.

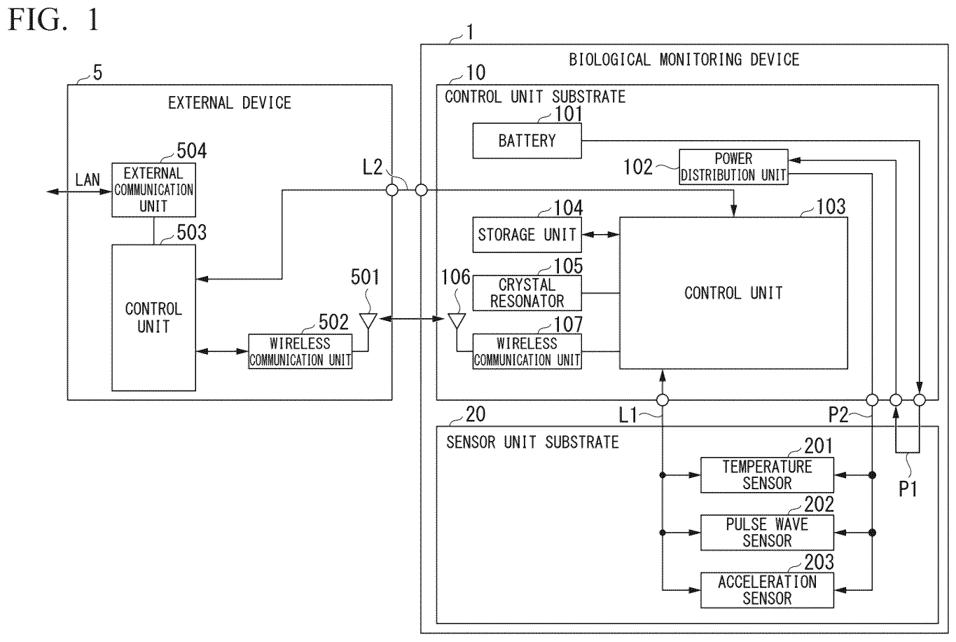

FIG. 1 is a block diagram showing an example of a configuration of a biological monitoring system according to one embodiment. In FIG. 1, the biological monitoring system includes a biological monitoring device 1 and an external device 5.

The biological monitoring device 1 and the external device 5 perform wireless communication. In addition, the biological monitoring device 1 and the external device S can perform wired communication via a signal line L2. Here, in FIG. 1, as an example of the connection of the signal line L2 between the biological monitoring device 1 and the external device 5, control units 103 and 503 are connected by the signal line L2, but the present invention is not limited thereto. For example, a storage unit 104 and a wireless communication unit 107 of the biological monitoring device 1 may be connected to the control unit 503 of the external device 5 via the signal line L2. In this case, the control unit 503 of the external device 5 may directly control the storage unit 104 and the wireless communication unit 107 of the biological monitoring device 1.

The biological monitoring device 1 includes a control unit substrate 10 and a sensor unit substrate 20. A battery 101, a power distribution unit 102, the control unit 103, the storage unit 104, a crystal resonator 105, an antenna 106 and the wireless communication unit 107 are mounted on the control unit substrate 10. A temperature sensor 201, a pulse wave sensor 202 and an acceleration sensor 203 are mounted on the sensor unit substrate 20. Hereinafter, the temperature sensor 201, the pulse wave sensor 202 and the acceleration sensor 203 will be referred to as a sensor 20X when they are not particularly distinguished.

In addition, a sensor-side connector CN1 (not shown in FIG. 1) is mounted on the sensor unit substrate 20, and a control unit-side connector CN2 (not shown in FIG. 1) is mounted on the control unit substrate 10. The sensor-side connector CN1 and the control unit-side connector CN2 are fitted together. The sensor unit substrate 20 and the control unit substrate 10 are detachable using the sensor-side connector CN1 and the control unit-side connector CN2. The sensor unit substrate 20 and the control unit substrate 10 are connected by fitting the sensor-side connector CN1 and the control unit-side connector CN2. When the sensor-side connector CN1 and the control unit-side connector CN2 are disconnected, the sensor unit substrate 20 and the control unit substrate 10 are disconnected.

The control unit-side connector CN2 and the sensor-side connector CN1 connect power source lines P1 and P2 and a signal line L1 between the side of the control unit substrate 10 and the side of the sensor unit substrate 20. Specifically, when the control unit-side connector CN2 and the sensor-side connector CN1 are fitted together, the power source lines P1 and P2 and the signal line L1 are connected between the control unit substrate 10 and the sensor unit substrate 20. On the other hand, when the control unit-side connector CN2 and the sensor-side connector CN1 are disconnected, the power source lines P1 and P2 and the signal line L1 are disconnected between the control unit substrate 10 and the sensor unit substrate 20.

The battery 101 is a power source that supplies power to respective units mounted on the control unit substrate 10 and the sensor 20X mounted on the sensor unit substrate 20. The power distribution unit 102 distributes the power supplied from the battery 101 to respective units mounted on the control unit substrate 10 and the sensor 20X mounted on the sensor unit substrate 20.

The biological monitoring device 1 in FIG. 1 supplies power to respective units mounted on the control unit substrate 10 and the sensor 20X mounted on the sensor unit substrate 20 only when the control unit-side connector CN2 and the sensor-side connector CN1 are fitted together. Specifically, as shown in FIG. 1, the power source line P1 for supplying power form the battery 101 is wired so that it passes once from the control unit substrate 10 through the sensor unit substrate 20 and is then connected to the power distribution unit 102 of the control unit substrate 10. Power source lines (not shown) are wired on the control unit substrate 10 from the power distribution unit 102 to respective units mounted on the control unit substrate 10. In addition, the power source line P2 is wired from the power distribution unit 102 to be connected from the control unit substrate 10 to the sensor 20X mounted on the sensor unit substrate 20.

When the power source lines P1 and P2 shown in FIG. 1 are wired in this manner, and thus the control unit-side connector CN2 and the sensor-side connector CN1 are fitted together, power is supplied to respective units mounted on the control unit substrate 10 through the power distribution unit 102 from the battery 101 via the power source line P1, and power is supplied to the sensor 20X mounted on the sensor unit substrate 20 via power source line P2. On the other hand, when the control unit-side connector CN2 and the sensor-side connector CN1 are disconnected, supply of power from the battery 101 to respective units mounted on the control unit substrate 10 and the sensor 20X mounted on the sensor unit substrate 20 is stopped. Therefore, since power consumed when the control unit-side connector CN2 and the sensor-side connector CN1 are disconnected is only naturally discharged, the effect of restricting wear of the battery 101 is obtained.

The sensor 20X operates by receiving power from the battery 101. The sensor 20X outputs a detection signal that it has detected. The temperature sensor 201 outputs a temperature detection signal indicating a temperature that it has detected. The pulse wave sensor 202 outputs a pulse wave detection signal indicating a pulse wave that it has detected. The acceleration sensor 203 outputs an acceleration detection signal indicating an acceleration that it has detected. A detection signal (sensor output signal) output from the sensor 20X is transmitted to the control unit 103 mounted on the control unit substrate 10 via the signal line L1.

The signal line L1 is a bidirectional serial communication type signal line including a transmission clock, a first signal and a second signal. The first signal is a signal that is transmitted from the sensor 20X to the control unit 103 in synchronization with the transmission clock. The second signal is a signal that is transmitted from the control unit 103 to the sensor 20X in synchronization with the transmission clock.

The control unit 103 controls the sensor 20X mounted on the sensor unit substrate 20 and the storage unit 104 and the wireless communication unit 107 mounted on the control unit substrate 10. The control unit 103 has preset measurement conditions. The control unit 103 sets the sensor 20X for measurement according to the measurement conditions. In addition, the control unit 103 samples a sensor output signal at a certain period according to the measurement conditions and stores the sampled sensor output signal in the storage unit 104 in association with a recording time. The recording time is a time measured by a clock unit. As an example, the recording time is in units of years, months, days, hours, minutes, and seconds. Here, the recording time is not limited to the time, and may be an index indicating the time elapsed from a reference time. For example, the recording time may indicate a time elapsed from when the control unit 103 starts.

The storage unit 104 stores various types of data such as a sensor output signal.

The crystal resonator 105 generates a fundamental frequency signal having a fundamental frequency for generating a clock used in the biological monitoring device 1. The fundamental frequency signal is used, for example, as an operating frequency of a clock of the control unit 103. The time of the clock of the control unit 103 is used, for example, as the recording time of the sensor output signal. Here, the clock of the control unit 103 is an example of the clock unit. That is, in the present embodiment, the clock unit is provided as a function of the control unit 103. The clock unit may be mounted on the control unit substrate 10 as a circuit separate from the control unit 103.

The control unit 103 wirelessly transmits the sensor output signal and recording time stored in the storage unit 104 to the external device 5 through the wireless communication unit 107. In addition, the control unit 103 wirelessly transmits an operation status signal indicating the operation status of the biological monitoring device 1 to the external device 5 through the wireless communication unit 107.

The wireless communication unit 107 transmits and receives radio signals via the antenna 106.

The external device 5 includes an antenna 501, a wireless communication unit 502, the control unit 503, and an external communication unit 504. The wireless communication unit 502 transmits and receives radio signals via the antenna 501. The wireless communication unit 107 of the biological monitoring device 1 and the wireless communication unit 502 of the external device 5 perform wireless communication using radio signals that are transmitted and received via the antennas 106 and 501.

The control unit 503 wirelessly receives the sensor output signal and the recording time from the biological monitoring device 1. In addition, the control unit 503 wirelessly receives the operation status signal from the biological monitoring device 1.

The control unit 503 performs a predetermined process on the received sensor output signal and recording time, and causes the external communication unit 504 to transmit the result of the process to a predetermined destination via a local area network (LAN). In addition, the control unit 503 performs a predetermined process on the received operation status signal, and causes the external communication unit 504 to transmit the result of the process to a predetermined destination via a local area network (LAN).

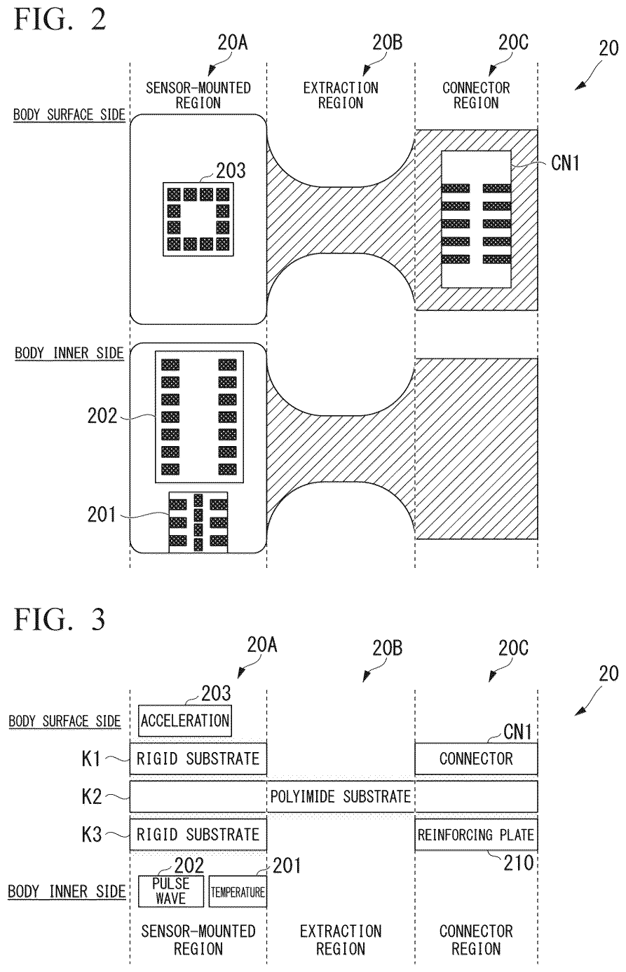

FIG. 2 is a plan view showing an example of a mounting configuration of the sensor unit substrate 20 according to the present embodiment. As shown in FIG. 2, the sensor unit substrate 20 has a sensor-mounted region 20A, an extraction region 20B, and a connector region 20C.

The sensor 20X is mounted in the sensor-mounted region 20A. The sensor-mounted region 20A is used by being implanted in the living body. The living body is a human or a non-human animal. The sensor-mounted region 20A is non-flexible.

Between both surfaces of the sensor-mounted region 20A, the temperature sensor 201 and the pulse wave sensor 202 are mounted on the surface that is inside the body when the sensor is implanted in the living body. The reason for mounting the temperature sensor 201 on the surface that is inside the body is so that it can measure a body temperature closer to a core body temperature compared to when the sensor is mounted on the body surface side and it is not easily influenced by the environment outside the body such as room temperature. The reason why the pulse wave sensor 202 is mounted on the surface that is inside the body is so that the influence of ambient light is reduced, the signal-to-noise ratio (SNR) is improved and the sensor is not influenced by the skin compared to when the sensor is mounted on the body surface side. On the other hand, between both surfaces of the sensor-mounted region 20A, the acceleration sensor 203 is mounted on the surface on the body surface side when the sensor is implanted in the living body. Here, one or more of the temperature sensor 201 and the pulse wave sensor 202 may be mounted on the body surface side.

Here, when the sensor 20X is implanted in the living body, compared to when it is disposed on the body surface, the sensor can be stably held at a desired measurement position, and thus stable measurement results can be obtained. In addition, when the acceleration sensor 203 is implanted in the living body, compared to when it is disposed on the body surface, it is not easily influenced by shock and vibration absorption by the skin, and thus the measurement accuracy of acceleration is improved.

As shown in FIG. 2, the ends of the sensor-mounted region 20A are chamfered. If the ends of the sensor-mounted region 20A are chamfered, when the sensor-mounted region 20A is implanted in the living body, body tissue is less likely to be damaged and the sensor-mounted region 20A can be inserted smoothly into the living body without being caught.

The extraction region 20B has the power source lines P1 and P2 and the signal line L1 between it and the control unit substrate 10. The extraction region 20B is used by being disposed across the inside of the living body in which the sensor-mounted region 20A is implanted and outside the living body. The extraction region 20B is flexible.

In the connector region 20C, the sensor-side connector CN1 is mounted. The sensor-side connector CN1 is a connector that fits into the control unit-side connector CN2 of the control unit substrate 10. The connector region 20C is used by being disposed outside the living body. The connector region 20C is non-flexible. In addition, the control unit substrate 10 is non-flexible.

As shown in FIG. 2, the extraction region 20B has a constricted shape having a width smaller than both ends on the side of the sensor-mounted region 20A and on the side of the connector region 20C. The constricted shape may be formed at any position in the extraction region 20B. As an example of the constricted shape, FIG. 2 shows a constricted shape in which the width of the intermediate part is smaller compared to both ends on the side of the sensor-mounted region 20A and on the side of the connector region 20C.

FIG. 3 is a cross-sectional view showing a example of a substrate configuration of the sensor unit substrate 20 according to the present embodiment. The sensor-mounted region 20A and the connector region 20C are non-flexible, and the extraction region 20B is flexible. A rigid-flexible substrate that can change the flexibility for each region is suitable for forming such a substrate.

In the present embodiment, as shown in FIG. 3, the sensor unit substrate 20 has a 3-layer substrate structure composed of a rigid substrate K1, a polyimide substrate K2, and a rigid substrate K3. In the sensor-mounted region 20A, a non-flexible substrate in which a base such as FR4 (the rigid substrates K1 and K3) is adhered to both surfaces of the polyimide substrate K2 is used. In the extraction region 20B, a flexible substrate composed of only the polyimide substrate K2 is used. In the connector region 20C, a polyimide substrate K2 with a reinforcing plate 210 is used.

According to the substrate configuration of the sensor unit substrate 20 shown in FIG. 3, it is possible to realize a configuration in which only the extraction region 20B is curved without damaging a solder mounting part of the sensor 20X or the sensor-side connector CN1. In addition, in order to shorten the length of the extraction region 20B as much as possible and crosslink from the subcutaneous layer to the epidermal surface, the extraction region 20B needs to have sufficient flexibility. Accordingly, the extraction region 20B is preferably a single-layer polyimide substrate composed of only the polyimide substrate K2.

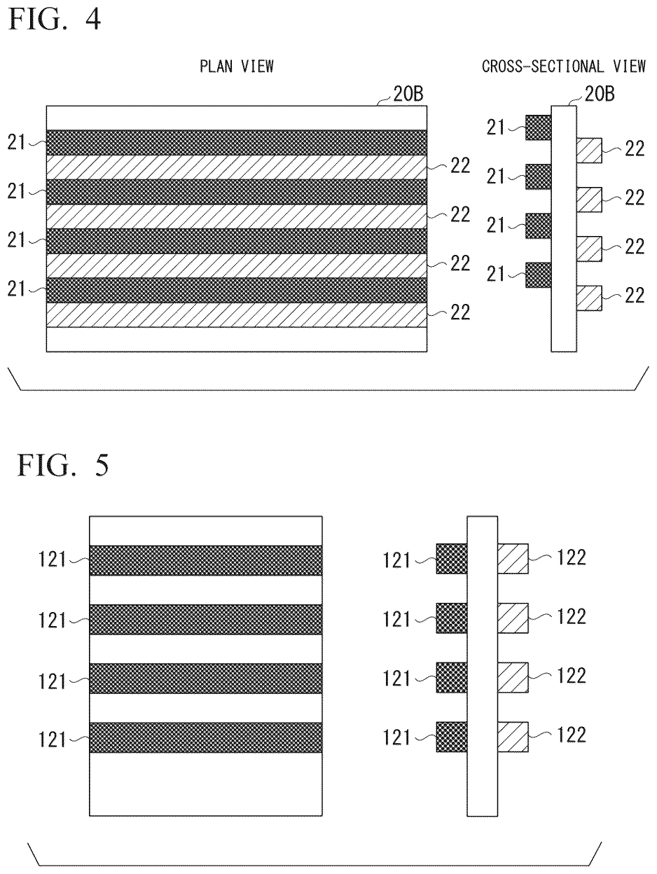

FIG. 4 show a plan view and a cross-sectional view showing a example of a substrate configuration of metal wires in the extraction region 20B according to the present embodiment. It is necessary to provide an electrical wire from the sensor-mounted region 20A to the connector region 20C using metal wires. It is preferable that the metal wires be alternately disposed on the front and back sides of the sensor unit substrate 20 so that the metal wires do not overlap each other in the same parts on the front and back sides. Front metal wires 21 are metal wires disposed on the front surface of the sensor unit substrate 20 in the extraction region 20B. Back metal wires 22 are metal wires disposed on the back surface of the sensor unit substrate 20 in the extraction region 20B. The plurality of front metal wires 21 and the plurality of back metal wires 22 are alternately disposed on the front and back sides of the sensor unit substrate 20 in the extraction region 20B so that they do not overlap each other in the same parts on the front and back sides. It is preferable that the thickness of the metal wire be sufficiently thin. In addition, it is preferable that the metal wire do not include a solid wire.

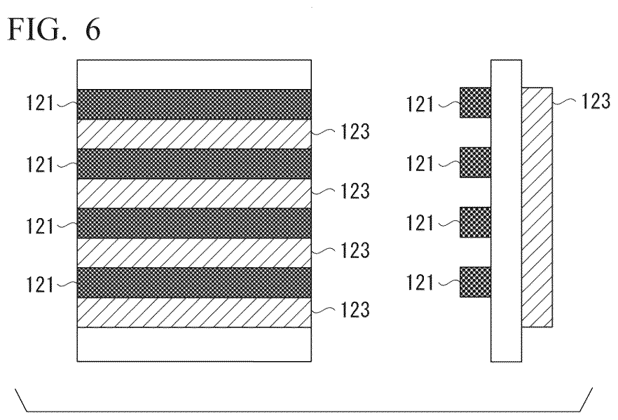

Here, for comparison with metal wires in the extraction region 20B according to the present embodiment, an example in which metal wires are not alternately disposed on the front and back sides of the sensor unit substrate 20 so that the metal wires do not overlap each other in the same parts on the front and back sides is shown. In metal wires shown in FIG. 5, a plurality of front metal wires 121 and a plurality of back metal wires 122 are disposed so that they overlap each other in the same parts on the front and back sides of the sensor unit substrate 20. In metal wires shown in FIG. 6, the plurality of front metal wires 121 and a back metal wire 123 are disposed so that they overlap each other in the same parts on the front and back sides of the sensor unit substrate 20. The back metal wire 123 has a metal pattern that spreads out in a flat shape.

In the biological monitoring device 1 according to the present embodiment, when the metal wires in the extraction region 20B are alternately disposed on the front and back sides of the sensor unit substrate 20 so that the metal wires do not overlap each other in the same parts on the front and back sides, the flexibility of the extraction region 20B can be increased compared to when the metal wires are disposed on the front and back sides of the sensor unit substrate 20 so that the metal wires overlap each other in the same parts. Here, the metal wires in the extraction region 20B may have some parts in which the metal wires overlap each other in the same parts on the front and back sides of the sensor unit substrate 20. However, in order to increase the flexibility of the extraction region 20B, it is preferable that the metal wires in the extraction region 20B not have parts in which the metal wires overlap each other in the same parts on the front and back sides of the sensor unit substrate 20.

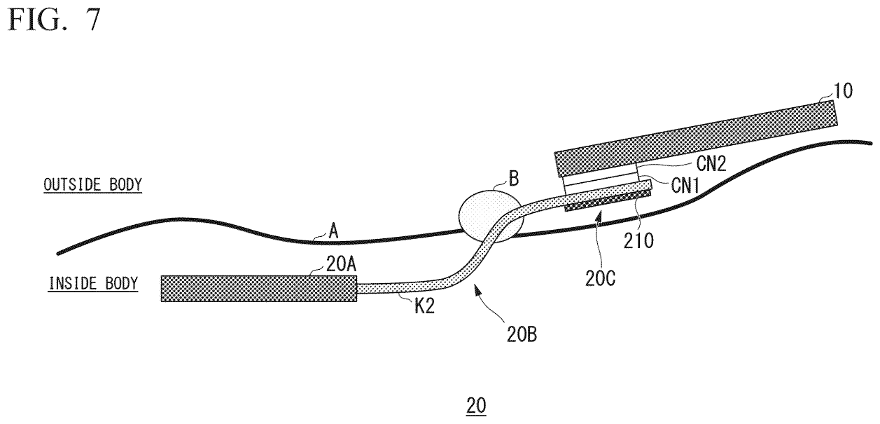

FIG. 7 is an illustrative diagram showing an example of usage of the biological monitoring device 1 according to the present embodiment. As shown in FIG. 7, the sensor-mounted region 20A of the sensor unit substrate 20 is implanted in the living body. In addition, the extraction region 20B of the sensor unit substrate 20 is disposed across the inside of the living body and the outside the living body. In addition, the connector region 20C of the sensor unit substrate 20 is disposed outside the living body.

The control unit substrate 10 is disposed outside the living body. Accordingly, the sensor-side connector CN1 is disposed on the side of the control unit substrate 10 of the sensor unit substrate 20 that is disposed outside the living body.

In the extraction region 20B, a part extending from the inside of the living body to the outside of the living body is curved to smoothly connect the sensor-mounted region 20A implanted in the living body to the connector region 20C disposed outside the living body. The sensor-side connector CN1 of the connector region 20C of the sensor unit substrate 20 is fitted into the sensor-side connector CN1 of the control unit substrate 10 disposed outside the living body.

The incised part of the skin A for inserting the sensor-mounted region 20A and the extraction region 20B into the living body is sutured or adhered with a medical adhesive, but a part B where the extraction region 20B exits the inside of the living body to the outside of the living body cannot be completely closed. If the wound at the part B is large, it may cause a high level of stress to the living body, it may interfere with normal activities of the living body, and biological information obtained by the sensor 20X may become ineffective for biological analysis of the living body. Accordingly, it is preferable that the wound at the part B be as small as possible.

As shown in FIG. 2, since the extraction region 20B according to the present embodiment has a constricted shape, the wound at the part B can be made smaller. Since only the power source lines P1 and P2 and the signal line L1 are mounted in the extraction region 20B, the minimum width of the constricted shape of the extraction region 20B can be made considerably smaller. When the narrowest part of the constricted shape is disposed at the part B, since the wound at the part B can be minimized, it is possible to minimize stress applied to the living body and to obtain an effect of inhibiting bacterial infection in the living body. In addition, when the extraction region 20B has a constricted shape, the constricted shape can be used as a guide for suturing. Therefore, an effect of preventing suturing from being performed while the sensor unit substrate 20 is shifted from a predetermined mounting position is obtained.

Considering application to small animals, the minimum width of the constricted shape of the extraction region 20B is, for example, preferably 5 millimeters (mm) or less. In addition, when the sensor-mounted region 20A is implanted in the subcutaneous layer of general people and animals, the region length of the extraction region 20B is preferably 3 mm to 10 mm. If the length of the extraction region 20B is too short, a load is applied to the side of the rigid substrates K1 and K3, and there is a risk of mounted components being damaged. On the other hand, if the length of the extraction region 20B is too long, fixation of the control unit substrate 10 becomes unstable, the control unit substrate 10 is positioned farther away by the length of the extraction region 20B, the moment of inertia observed from the sensor unit substrate 20 increases, and thus the sensor unit substrate 20 is more likely to move significantly when the living body moves. This causes the part measured by the sensor 20X to shift and thus it is not possible to perform stable measurement.

Since the sensor unit substrate 20 is disposed in the living body, waterproofing with a parylene coating is performed. Here, since only minimum necessary components such as the sensor 20X that need to be implanted in the living body are mounted on the sensor unit substrate 20, the amount of unevenness is reduced, and coating coverage is improved. Therefore, an effect of forming a high-quality waterproof film is obtained.

On the other hand, since the control unit substrate 10 is disposed outside the living body, excessive waterproofing is not necessary. This is advantageous when components for which electrical contacts such as battery holders and connectors need to be provided are disposed, and a user can replace the battery 101 and perform connection to the external device 5 using the control unit-side connector CN2 without any special fixing.

On the control unit substrate 10 disposed outside the living body, the control unit 103, the storage unit 104, the wireless communication unit 107 and the like, which do not need to be disposed in the living body, are mounted, but particularly when the wireless communication unit 107 is disposed in the living body, since radio wave transmission characteristics deteriorate due to radio wave absorption by body tissue, it is preferable to dispose the wireless communication unit 107 outside the living body. In addition, compared to when the sensor is installed in the living body, opportunities for contact with the body fluid are reduced, and thus corrosion can be reduced and short-circuiting due to water and increased current consumption can be prevented.

In addition, when the control unit substrate 10 is detachable from the sensor unit substrate 20 using the sensor-side connector CN1 and the control unit-side connector CN2, only the control unit substrate 10 can be removed while the sensor unit substrate 20 remains attached to the living body. Therefore, for example, when the capacity of the battery 101 is insufficient or the free space of the storage unit 104 is insufficient, it is easy to replace only the control unit substrate 10. In addition, since it is not necessary to remove the sensor unit substrate 20 implanted in the living body from the living body and implant it again in the living body, the burden on the living body is significantly reduced.

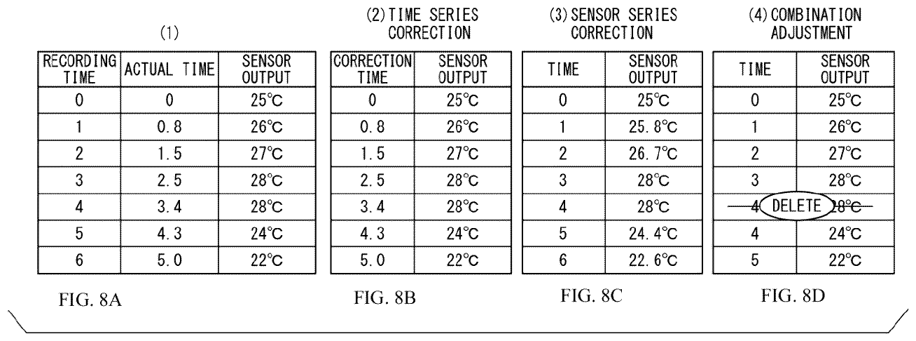

FIGS. 8A, 8B, 8C and 8D are diagrams showing an example of processing a sensor output signal according to the present embodiment. Here, the values shown in FIGS. 8A-8B are values for convenience of explanation.

In the example in FIGS. 8A-8D, the sensor output signal is a temperature detection signal of the temperature sensor 201. As shown in FIG. 8A, the recording time recorded in the storage unit 104 of the biological monitoring device 1 in association with the sensor output signal differs from the actual time. This is due to factors such as the frequency accuracy of the crystal resonator 105 that generates the original fundamental frequency signal for the operating frequency of the clock of the control unit 103 of the biological monitoring device 1. The biological monitoring device 1 itself cannot determine this time difference, but when information is periodically transmitted from the wireless communication unit 107 of the biological monitoring device 1, the external device 5 which receives the information through the wireless communication unit 502 can subsequently correct the time.

Hereinafter, an example of a method of processing a sensor output signal according to the present embodiment will be described.

The control unit 103 of the biological monitoring device 1 causes the wireless communication unit 107 to wirelessly transmit a predetermined signal to the external device 5 at a predetermined transmission period (here, a 10-second period as an example) based on the time of its own clock (that is, a time measured by the clock unit). The control unit 503 of the external device 5 uses its own clock to record a reception time of the predetermined signal that is wirelessly received through the wireless communication unit 502. The clock of the control unit 503 of the external device 5 is, for example, a clock that keeps accurate time using a predetermined time correction method such as a radio clock. The control unit 503 calculates, based on the record of the reception time of the predetermined signal, the difference (time difference) between the “10-second period” as the transmission period of the biological monitoring device 1 and the 1-second period based on the time of its own clock.

The control unit 103 of the biological monitoring device 1 causes the wireless communication unit 107 to wirelessly transmit the sensor output signal and the recording time stored in the storage unit 104 to the external device 5. As described above, the recording time is associated with the sensor output signal. The control unit 503 of the external device 5 corrects time series data of the sensor output signal and the recording time wirelessly received through the wireless communication unit 502 based on the time difference. Consequently, the external device 5 corrects time series data of the sensor output signal and the recording time wirelessly received by the external device 5 based on the reception time of the predetermined signal wirelessly received by the external device 5.

FIG. 8B shows a correction example 1 of time series data of the sensor output signal and the recording time. In the correction example 1 shown in FIG. 8B, in the time series data of the sensor output signal and the recording time, the recording time is corrected based on the time difference. In this correction method, for example, the time difference may be directly reflected in the recording time or the difference of the recording time is predicted according to the change in time difference, and the recording time may be corrected based on the prediction. In addition, when the time difference calculation interval is long, the time difference calculation result may be interpolated using linear interpolation or the like. According to the correction example 1 shown in FIG. 8B, the corrected recording time (correction time) associated with the sensor output signal is adjusted to the actual time.

FIG. 8C shows a correction example 2 of time series data of the sensor output signal and the recording time. In the correction example 2 shown in FIG. 8C, in the time series data of the sensor output signal and the recording time, the sensor output signal is corrected based on the time difference. In this correction method, the sensor output signal corresponding to the actual time is interpolated so that the time series data has the same time interval as the recording time.

FIG. 8D shows a correction example 3 of time series data of the sensor output signal and the recording time. In the correction example 3 shown in FIG. 8D, in the time series data of the sensor output signal and the recording time, correction is performed for each combination of the sensor output signal and the recording time based on the time difference. This correction method is a method of deleting or adding a combination of a sensor output signal and a recording time when it is not desired to directly operate the recording time and the sensor output signal. For example, if a time difference of one or more increments occurs when the actual time is rounded, dummy data is inserted or existing data is deleted to adjust the time series data to have a plausible combination of the time and the sensor output signal.

The above is an explanation of an example of a method of processing a sensor output signal.

In addition, the external device 5 may read out at least some sensor output signals stored in the storage unit 104 of the biological monitoring device 1 from the biological monitoring device 1 via wired connection or wireless connection after the biological monitoring device 1 has completed the measurement, rather than immediately determining the wirelessly transmitted sensor output signal. In this case, transmitting all sensor output signals via wireless transmission is not preferable in terms of power consumption and radio wave resource consumption. Thus, for example, instead of a sensor output signal, an identifier (ID) associated with the sensor output signal may be transmitted in combination with the recording time. In this case, after the biological monitoring device 1 has completed the measurement, the external device 5 may correct the recording time based on the association.

In addition, as described above, the control unit 103 may cause the wireless communication unit 107 to wirelessly transmit a signal indicating the operation status of the biological monitoring device 1 (operation status signal) to the external device 5. The operation status signal may include a voltage of the battery 101, a sensor output signal (or an outline of the sensor output signal), a recording time (or information indicating the time), and an identifier of the biological monitoring device 1. When the external device 5 receives the operation status signal, the operation statuses of the plurality of biological monitoring devices 1 can be centrally managed. Therefore, the user can determine the operation status of the biological monitoring device 1. The operation status of the biological monitoring device 1 includes, for example, when the battery should be replaced or whether the device is operating normally.

In addition, the control unit 103 may notify the user of whether the installation position of the sensor 20X is normal. For the notification, the sensor output signal is used. The control unit 103 causes the wireless communication unit 107 to wirelessly transmit the sensor output signal to the external receiver. The receiver presents an installation position evaluation index based on the sensor output signal. The installation position evaluation index is an index for evaluating whether the installation position of the sensor 20X is normal.

For example, when the sensor 20X is an optical sensor, the installation position evaluation index indicates that the installation position of the sensor 20X is normal when the light intensity value (each of the DC component and the AC component) falls within a specific range, and indicates that the installation position of the sensor 20X is not normal when the light intensity value does not fall within a specific range. For example, when the sensor 20X is an acceleration sensor, the installation position evaluation index indicates that the installation position of the sensor 20X is normal when gravitational acceleration occurs in a specific direction, and indicates that the installation position of the sensor 20X is not normal when gravitational acceleration does not occur in a specific direction.

The sensor 20X for installation position evaluation includes, for example, a temperature sensor, an acceleration sensor, an optical sensor, a pulse wave sensor, a blood oxygen saturation (SpO2) sensor, a nerve potential sensor, a brain potential sensor, and a muscle potential sensor.

The receiver is, for example, the external device 5. Here, the receiver may be a dedicated device for notifying the user of the installation position evaluation index.

The device communicates wirelessly with the biological monitoring device 1. The receiver may be a smartphone, a tablet or the like.

The receiver notifies the user of the installation position evaluation index by displaying the installation position evaluation index on a display device using, for example, text or an image such as a mark. The display device is provided in the receiver. The receiver may notify the user of the installation position evaluation index using light or sound. When light is used to notify of the installation position evaluation index, the receiver includes, for example, a light emitting unit including a LED lamp. When a sound is used to notify of the installation position evaluation index, the receiver includes, for example, a speaker.

Here, the receiver may notify the user of the installation position evaluation index only when the installation position is not normal.

As described above, based on the sensor output signal received by the external receiver, the user can know whether the installation position of the sensor 20X is normal. For example, the user can infer whether the position where the sensor 20X is implanted in the subcutaneous layer is appropriate, whether the sensor 20X implanted in the subcutaneous layer is floating in the subcutaneous layer, whether the installation position is shifted due to a mixed in foreign substance, and whether the sensor 20X is tilted. The user can correct the installation position of the sensor 20X when the installation position is not normal. Consequently, it is possible to prevent the measurement accuracy of the sensor 20X from decreasing due to the shift in the installation position.

Here, instead of the external receiver that receives the sensor output signal, a notification unit may be provided on the control unit substrate 10 of the biological monitoring device 1. The notification unit notifies the user of whether the installation position of the sensor 20X is normal. The notification unit notifies the user of the installation position evaluation index using, for example, light or sound. The notification unit includes, for example, an LED lamp or a speaker.

The control unit-side connector CN2 of the control unit substrate 10 of the biological monitoring device 1 according to the present embodiment may also be used for connection to the external device 5. Therefore, the control unit substrate 10 and the external device 5 can be connected via the control unit-side connector CN2. The control unit-side connector CN2 connects the signal line L2 shown in FIG. 1 between the control unit substrate 10 and the external device 5. The control unit 103, the storage unit 104 and the wireless communication unit 107 mounted on the control unit substrate 10 are operable from the external device 5 connected to the control unit substrate 10 via the control unit-side connector CN2. Specifically, the control unit 503 of the external device 5 transmits and receives a control signal to and from the control unit 103 mounted on the control unit substrate 10 via the signal line L2. Therefore, the control unit 503 of the external device 5 accesses the control unit 103 mounted on the control unit substrate 10, and additionally accesses the storage unit 104 and the wireless communication unit 107 via the control unit 103.

For example, the control unit 503 of the external device 5 sets measurement conditions and the like for the biological monitoring device 1. For example, the control unit 503 of the external device 5 reads out the measurement data (the sensor output signal, the recording time, etc.) recorded in the storage unit 104 of the biological monitoring device 1 at a high speed through wired communication via the signal line L2. For example, the control unit 503 of the external device 5 performs a test of the wireless communication unit 107 of the biological monitoring device 1 and the like.

In addition, the sensor-side connector CN1 of the sensor unit substrate 20 of the biological monitoring device 1 according to the present embodiment may also be used for connection to the external device 5. Therefore, the sensor unit substrate 20 and the external device 5 can be connected via the sensor-side connector CN1. The sensor-side connector CN1 connects the signal line L1 shown in FIG. 1 between the sensor unit substrate 20 and the external device 5. The sensor 20X mounted on the sensor unit substrate 20 is operable from the external device 5 connected to the sensor unit substrate 20 via the sensor-side connector CN1. Specifically, the control unit 503 of the external device 5 transmits and receives a control signal to and from the sensor 20X mounted on the sensor unit substrate 20 via the signal line L1. Therefore, the control unit 503 of the external device 5 may perform calibration, a self-test and the like of the sensor 20X mounted on the sensor unit substrate 20.

Here, in the present embodiment, an example in which the sensor-side connector CN1 and the control unit-side connector CN2 mounted in the connector region 20C are fitted together to connect the extraction region 20B of the sensor unit substrate 20 to the control unit substrate 10 has been described. That is, an example in which the extraction region 20B and the control unit substrate 10 are connected via a connector bas been described, but the present invention is not limited thereto.

The extraction region 20B and the control unit substrate 10 may be connected without a connector. In this case, the extraction region 20B and the control unit substrate 10 are directly connected via a power source line and a signal line. In this case, the sensor unit substrate 20 may not have the connector region 20C.

In addition, at least one of the sensor unit substrate 20 and the control unit substrate 10 may have a connector region that fits into a connector. When the sensor unit substrate 20 has a connector region (the connector region 20C) that fits into a connector (the sensor-side connector CN1), as described in the embodiment, the sensor unit substrate 20 is connected to the control unit substrate 10 via the connector. When the control unit substrate 10 has a connector region that fits into a connector (the control unit-side connector CN2), the control unit substrate 10 is connected to the extraction region 20B via the connector.

When at least one of the sensor unit substrate 20 and the control unit substrate 10 has a connector region that fits into a connector, at least one of the sensor unit substrate 20 and the control unit substrate 10 is easily replaced.

Here, one or more sensors may be mounted on the sensor unit substrate 20.

That is, one or more of the temperature sensor 201, the pulse wave sensor 202 and the acceleration sensor 203 may be mounted on the sensor unit substrate 20. In addition, in the above embodiment, examples of types of sensors include the temperature sensor 201, the pulse wave sensor 202 and the acceleration sensor 203, but various sensors other than these sensors may be applied.

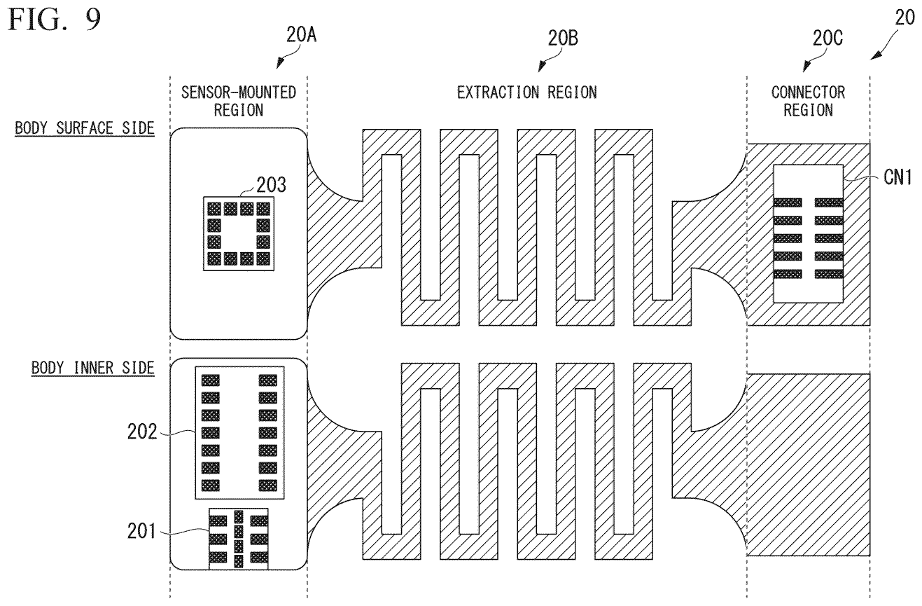

In addition, the extraction region 20B of the sensor unit substrate 20 may have any shape other than the above constricted shape exemplified in FIG. 2. FIG. 9 is a plan view showing an example of a mounting configuration of a sensor unit substrate. FIG. 9 shows an example of the shape of the extraction region 20B of the sensor unit substrate 20. In the example in FIG. 9, the extraction region 20B has a meander shape formed by a plurality of continuous curved parts. When the extraction region 20B has such a shape, the movement (for example, the force caused by body movement or vibration of the living body or the movement caused by the moment) of the control unit substrate 10 is buffered by the extraction region 20B and is less likely to be transmitted to the sensor-mounted region 20A. Therefore, vibrations and position shifts in the sensor-mounted region 20A are restricted, and an effect of preventing noise and artifacts from occurring in the detection signal (sensor output signal) output from the sensor 20X is obtained.

Here, the curved part may be curved at a right angle as in the example shown in FIG. 9 or may be curved in an arc shape. In addition, in the example in FIG. 9, the extraction region 20B has a plurality of constricted shapes in a direction in which the extraction region 20B extends. The direction in which the extraction region 20B extends is a direction from the sensor-mounted region 20A toward the connector region 20C. In other words, the direction in which the extraction region 20B extends is a wire direction of power source lines and signal lines. In addition, although the curved parts are continuous in the example in FIG. 9, the curved parts may not be continuous, and straight parts may be present between the curved parts. In addition, the number of curved parts may be one or plural.

It is preferable to provide a plurality of curved parts. As described above, the incised part for inserting the sensor-mounted region 20A and the extraction region 20B into the living body is sutured. The plurality of curved parts allow the position of the sensor-mounted region 20A and the position of the part to be sutured (referred to as a suture position) to be adjusted during suturing. If the relationship between the position of the part B (refer to FIG. 7) where the extraction region 20B exits the inside of the living body to the outside of the living body and the suture position is not determined in advance, the position of the part B may be shifted from the suture position. If there is only one curved part, it is difficult to adjust the position of the part B when the position of the part B is shifted from the suture position.

In addition, both end parts and the periphery of the extraction region 20B may have a linear shape. When both end parts and the periphery of the extraction region 20B have a linear shape rather than a meander shape, since the tension applied to each connection part with the sensor-mounted region 20A and the connector region 20C can be restricted, the durability of the connection parts is improved.

Here, when the extraction region 20B is formed in a meander shape, the radius of curvature of the curved part is preferably 1 mm to 2 mm. When the curved part is curved in an arc shape, the radius of curvature of the curved part is the radius of curvature of the arc. When the curved part is curved at a right angle, the radius of curvature of the curved part is, for example, the radius of curvature of an arc when the curved part is approximated by the arc.

In addition, in order to provide sufficient elasticity, the period of continuous curved parts is preferably smaller than the width of the curved part. The period of continuous curved parts is the distance between adjacent curved parts among the plurality of curved parts that are repeatedly disposed. The width of the curved part is the length from one end to the other end of the curved part in a direction perpendicular to the direction in which the plurality of curved parts are repeatedly disposed. In other words, the width of the curved part is the width of the curved part in the vertical direction in FIG. 9. For example, the period of continuous curved parts is 5 mm, and the width of the curved part is 8 mm.

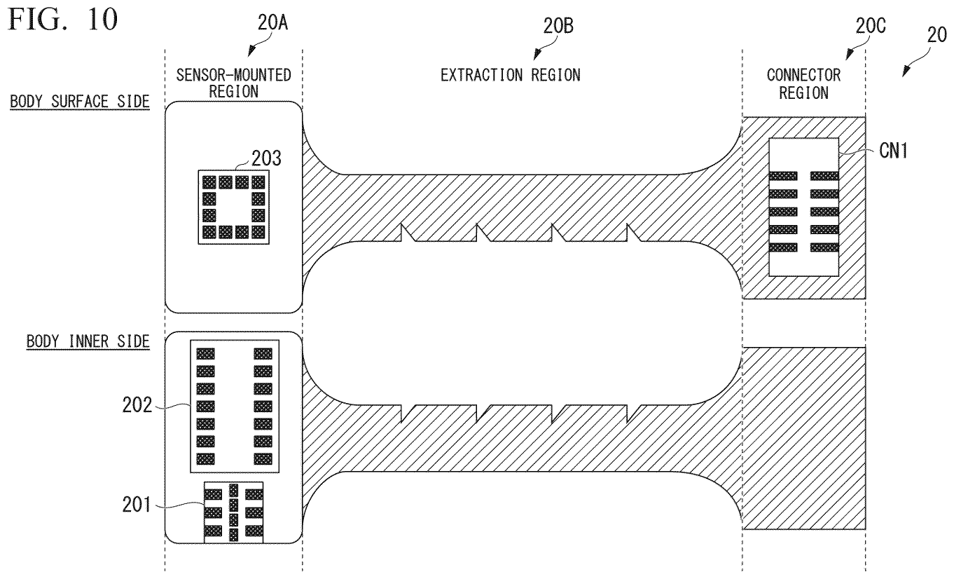

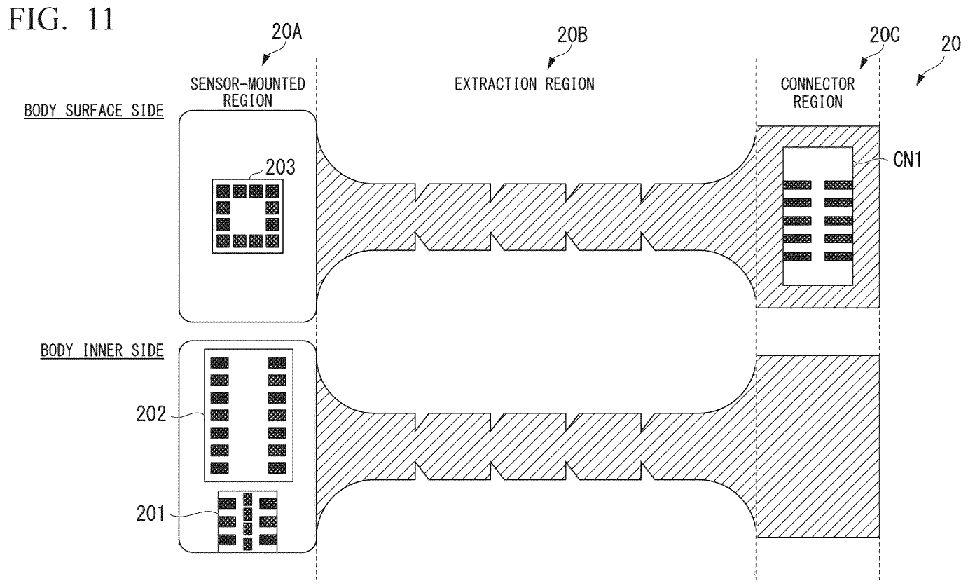

In addition, the extraction region 20B may have a wedge shape. FIG. 10 is a plan view showing an example of a mounting configuration of the sensor unit substrate 20. FIG. 10 shows an example of the shape of the extraction region 20B of the sensor unit substrate 20. In the example in FIG. 10, the extraction region 20B has a wedge shape on one side. In addition, FIG. 11 is a plan view showing an example of a mounting configuration of the sensor unit substrate 20. FIG. 11 shows an example of the shape of the extraction region 20B of the sensor unit substrate 20. In the example in FIG. 11, the extraction region 20B has a wedge shape on both sides.

Because the extraction region 20B has a wedge shape, even if a force is applied to the extraction region 20B, the connector region 20C, or the control unit substrate 10 from the outside toward the outside of the living body, the sensor-mounted region 20A is less likely to fall off of the living body.

In addition, it is preferable that either the sensor-mounted region 20A or the connector region 20C, or both the sensor-mounted region 20A and the connector region 20C have a displacement prevention unit. The displacement prevention unit prevents the position of the sensor unit substrate 20 from being shifted. As will be described below, the displacement prevention unit is composed of a protrusion, a through-hole or the like. The displacement prevention unit can prevent the position of the sensor-mounted region 20A in the subcutaneous layer from being shifted from the initial position at which the sensor-mounted region 20A is inserted into the living body, which causes noise in the sensor output signal, measurement fluctuation, or operation failure. In addition, as described above, the movement of the connector region 20C due to the movement of the control unit substrate 10 disposed outside the living body can be transmitted as vibration and movement to the sensor-mounted region 20A. Vibrations and position shifts in the sensor-mounted region 20A influence the sensor output signal. Consequently, when the displacement prevention unit is provided in at least one of the sensor-mounted region 20A and the connector region 20C, movement caused by body movement can be restricted in both the sensor-mounted region 20A and the connector region 20C. In addition, when the displacement prevention unit is a protrusion, the position of the sensor unit substrate 20 can be more firmly fixed by inserting the protrusion into the slit provided in the skin. The protrusion preferably has a size of about 1 mm to 2 mm.

Hereinafter, an example in which the sensor unit substrate 20 has a displacement prevention unit in the sensor-mounted region 20A will be described.

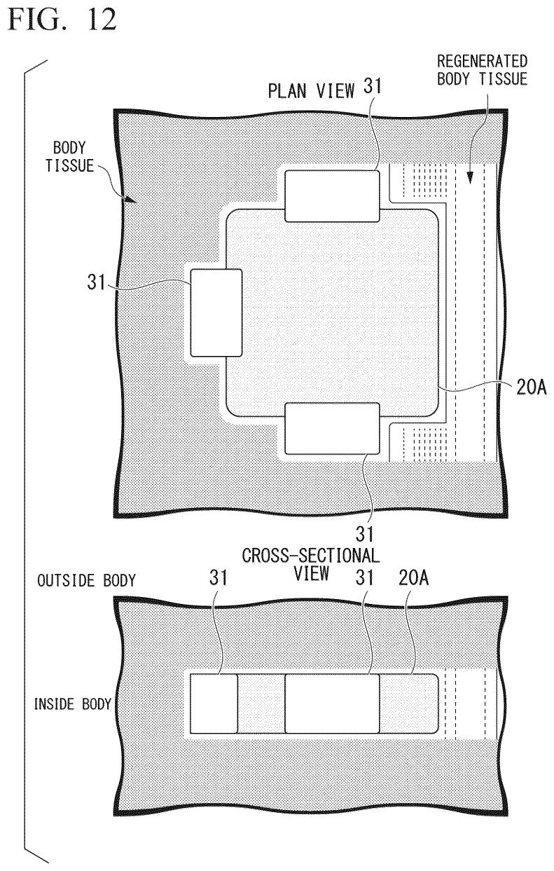

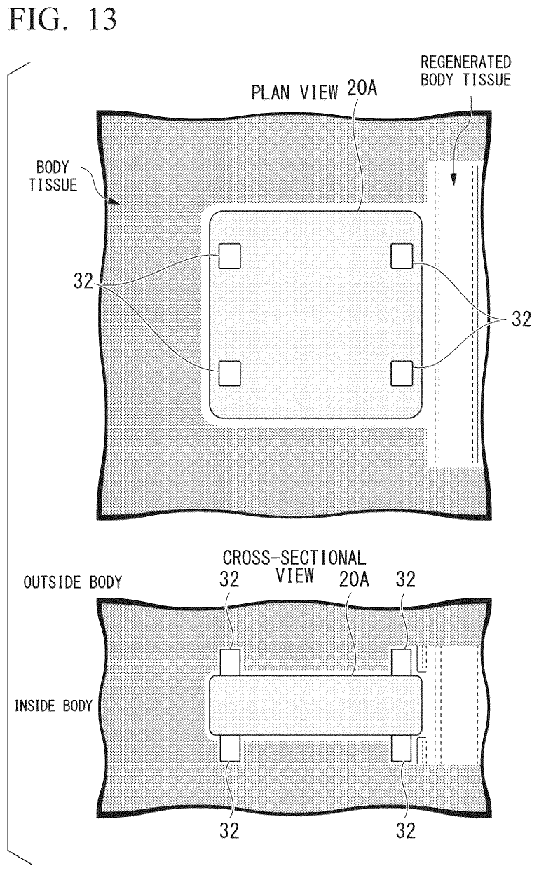

In order to prevent the position of the sensor-mounted region 20A implanted in the subcutaneous layer (that is, inside the living body) from being unintentionally shifted in the subcutaneous layer, the phenomenon in which the subcutaneous tissue regenerates and adheres due to natural healing power can also be used. For example, with the configuration shown in the following FIG. 12 to FIG. 14, it is possible to prevent the position of the sensor-mounted region 20A implanted in the subcutaneous layer from being unintentionally shifted in the subcutaneous layer.

FIG. 12 shows a plan view and a cross-sectional view showing an example of a mounting configuration of the displacement prevention unit. In the example shown in FIG. 12, the displacement prevention unit has protrusions 31. Three protrusions 31 are provided on three surfaces that form the outer periphery of the sensor-mounted region 20A. That is, the displacement prevention unit is positioned on the outer periphery of the sensor-mounted region 20A. The protrusion 31 protrudes from the surface toward the outside of the sensor-mounted region 20A. The height of the sensor-mounted region 20A and the height of the protrusion 31 are approximately equal. The upper surface of the protrusion 31 and the upper surface of the sensor-mounted region 20A are in the same plane. The bottom surface of the protrusion 31 and the bottom surface of the sensor-mounted region 20A are in the same plane.

Since the protrusion 31 is caught in the subcutaneous tissue, the position of the sensor-mounted region 20A is less likely to be shifted. In addition, since the subcutaneous tissue regenerates according to the shape of the sensor-mounted region 20A, the position of the sensor-mounted region 20A is less likely to be shifted after healing. When the displacement prevention unit is provided on the outer periphery of the sensor-mounted region 20A, since a large moment is less likely to occur compared to when the displacement prevention unit is provided on the inner periphery of the sensor-mounted region 20A, it becomes difficult for the sensor-mounted region 20A to rotate within the upper surface (or the bottom surface) of the sensor-mounted region 20A.

Here, the displacement prevention unit may have a recess (dent). For example, one or more of the protrusions 31 shown in FIG. 12 may be realized by a recess provided at an end of the base shape of the sensor-mounted region 20A.

In addition, the displacement prevention unit may be positioned at a corner that constitutes the outer periphery of the sensor-mounted region 20A.

FIG. 13 shows a plan view and a cross-sectional view showing an example of a mounting configuration of a displacement prevention unit. In the example shown in FIG. 13, the displacement prevention unit has protrusions 32. The protrusion 32 protrudes from both the front surface and the back surface of the sensor-mounted region 20A toward the outside of the sensor-mounted region 20A. That is, the displacement prevention unit is disposed substantially perpendicular to the interface between the epidermis and the subcutaneous layer. Four protrusions 32 are provided on each of the front surface and the back surface of the sensor-mounted region 20A. That is, a total of eight protrusions 32 are provided. The protrusions 32 are provided near four comers of each of the front surface and the back surface of the sensor-mounted region 20A. Since the subcutaneous tissue regenerates according to the shape of the protrusion 32, after healing, the position of the sensor-mounted region 20A is less likely to be shifted in a direction approximately parallel to the surface of the epidermis.

Here, the displacement prevention unit may not be disposed substantially perpendicular to the interface between the epidermis and the subcutaneous layer. The displacement prevention unit may be disposed at a predetermined angle tilted in a direction substantially perpendicular to the interface between the epidermis and the subcutaneous layer.

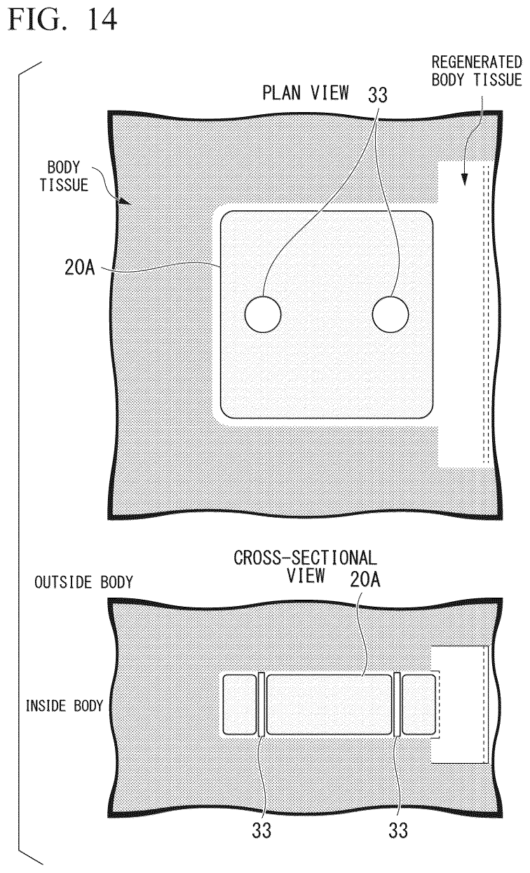

FIG. 14 shows a plan view and a cross-sectional view showing an example of a mounting configuration of a displacement prevention unit. In the example shown in FIG. 14, the displacement prevention unit has a hole. The shape of the hole is, for example, a circle. In the example shown in FIG. 14, two through-holes 33 are provided so that they penetrate from the front surface to the back surface of the sensor-mounted region 20A.

After the sensor-mounted region 20A is inserted into the living body, the subcutaneous tissue that regenerates according to the shape of the through-hole 33 is adhered to the through-hole 33, and the position of the sensor-mounted region 20A is less likely to be shifted. Providing holes as the displacement prevention units allows costs to be reduced by the cost of the protrusion material compared to providing protrusions, and holes are easier to produce than protrusions.

Here, the shape of the hole in the displacement prevention unit is not limited to a circle. The shape of the hole may be any shape such as a rectangle. In addition, the hole in the displacement prevention unit may be a non-through hole or a dent.

In addition, the displacement prevention unit may also have a protrusion and a hole. For example, one or more of the protrusions 32 shown in FIG. 13 may be replaced with non-through holes or dents. One or more of the through-holes 33 shown in FIG. 14 may be replaced with protrusions. That is, the displacement prevention unit may be formed as unevenness provided on the base of the sensor-mounted region 20A. The subcutaneous tissue regenerates according to the shape of these unevennesses. In the initial period after the sensor-mounted region 20A is inserted into the living body, the protrusions mainly prevent the position of the sensor-mounted region 20A from being shifted. Over time, the subcutaneous tissue regenerates, and after healing, the regenerated subcutaneous tissue adheres to holes, and thus an effect of preventing the position of the sensor-mounted region 20A from being shifted due to adhesion to the holes after healing is improved.

Here, the shift in the position of the sensor-mounted region 20A is, in other words, the shift in the installation position of the sensor 20X. The shift in the position of the sensor-mounted region 20A includes a shift in the position in a direction approximately parallel to the epidermal surface and a shift due to rotation within a plane approximately parallel to the epidermal surface.

As described above, the sensor unit substrate 20 has a plurality of displacement prevention units. In the sensor unit substrate 20, the plurality of displacement prevention units may be formed with one type of shape among various shapes (protrusions, holes, etc.) described above or formed with a plurality of types of shapes. Here, the sensor unit substrate 20 may have only one displacement prevention unit.

It is preferable that a sufficient number of displacement prevention units be provided at appropriate positions in the sensor-mounted region 20A in order to prevent the position of the sensor-mounted region 20A from being shifted under conditions that they do not overlap the position of the sensor 20X mounted in the sensor-mounted region 20A and the outer shape of the sensor-mounted region 20A itself does not become too complicated.

When the sensor unit substrate 20 has a plurality of displacement prevention units, it is preferable that the sensor unit substrate 20 have a plurality of displacement prevention units at positions that do not overlap when the sensor unit substrate 20 is viewed from above. For example, in the above examples shown in FIG. 12, FIG. 13, and FIG. 14, the plurality of displacement prevention units are provided at positions that do not overlap when the sensor-mounted region 20A is viewed from above. When the plurality of displacement prevention units are provided at positions that do not overlap when the sensor-mounted region 20A is viewed from above, rotation of the sensor-mounted region 20A around a certain displacement prevention unit within a plane when the sensor-mounted region 20A is viewed from above, is interfered with by another displacement prevention unit. That is, the plurality of displacement prevention units can prevent rotation of the sensor-mounted region 20A within a plane when the sensor-mounted region 20A is viewed from above.

In addition, the displacement prevention unit may be a part that has been subjected to a surface treatment in order to prevent the position of the sensor-mounted region 20A in the living body from being shifted. For example, the displacement prevention unit may be a fine unevenness provided on at least a part of the surface of the sensor-mounted region 20A. The fine unevenness is, for example, an unevenness having a height or depth of 10 micrometers to less than 1 millimeter from the surface of the sensor-mounted region 20A. The fine unevenness increases the resistance of the surface of the sensor-mounted region 20A and prevents the position of the sensor-mounted region 20A from being shifted.

In addition, the displacement prevention unit may be a part where at least a part of the surface of the sensor-mounted region 20A has been surface-treated with a chemical substance that promotes adhesion to biological tissue. For example, the surface of the sensor-mounted region 20A may be coated with a bioadhesive material such as a cyanoacrylate-based, biopolymer-aldehyde-based, or fibrin-based material, a hydrogel material formed of collagen, hyaluronic acid, alginate, chitosan, or silk fibroin, a bioactive ceramic material such as hydroxyapatite, bioglass, calcium phosphate, or a carbonate-containing apatite, or a porous material. The chemical substance that promotes adhesion to biological tissue makes it easier for the surface of the sensor-mounted region 20A to adhere to biological tissue, and an effect of fixation by adhesion can be improved.

In addition, in the case of transmission windows of the optical sensors such as the temperature sensor 201 and the pulse wave sensor 202, the optical characteristics may change due to adhesion of biological tissue, and there is a risk of the sensitivity of the optical sensor decreasing. Accordingly, it is effective not to apply a coating with a chemical substance to the part where there is a risk of the sensitivity of the optical sensor decreasing. In addition, an anti-adhesion agent may be applied to the periphery of the sensor 20X in the sensor-mounted region 20A. Examples of anti-adhesion agents include a gelling agent made of surface-modified gelatin, oxidized cellulose, sodium hyaluronate or carboxymethylcellulose, a dextrin-based anti-adhesion material, and a bio-inert ceramic material such as alumina. The anti-adhesion agent can prevent a decrease in sensitivity of the optical sensor caused by adhesion of biological tissue.

In addition, the displacement prevention unit may be made of a bioabsorbable material. For example, the protrusion as the displacement prevention unit may be made of a bioabsorbable material. When the displacement prevention unit is made of a bioabsorbable material, after the sensor-mounted region 20A is inserted into the living body, the displacement prevention unit is absorbed and disappears in the living body, and biological tissue regenerates in the part where the displacement prevention unit is provided. When the sensor-mounted region 20A is fixed with the regenerated biological tissue, the position of the sensor-mounted region 20A is prevented from being shifted.

When the displacement prevention unit is made of a bioabsorbable material, in the initial period after the sensor-mounted region 20A is inserted into the living body, the displacement prevention unit prevents the position of the sensor-mounted region 20A from being shifted, and after the subcutaneous tissue heals, biological tissue regenerates in the part where the displacement prevention unit is provided, and thus an effect of preventing the position of the sensor-mounted region 20A from being shifted is improved. Consequently, when the displacement prevention unit is made of a bioabsorbable material, the same effect as that obtained when the above displacement prevention unit has both a protrusion and a hole can be obtained. In addition, since the bioabsorbable material is absorbed and disappears in the living body, the volume that the sensor-mounted region 20A occupies in the living body can be reduced and the burden on the living body into which the sensor is implanted can be reduced.

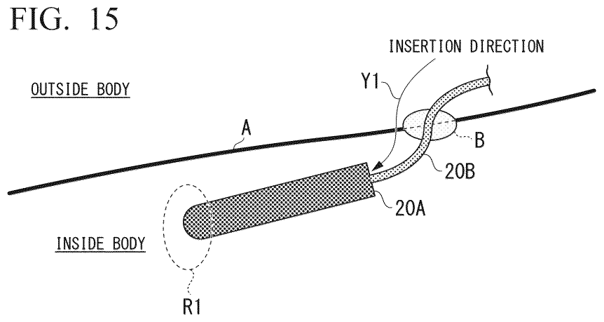

In addition, the sensor-mounted region 20A may have a rounded shape on the side opposite to the extraction region 20B. Here, as shown in FIG. 15, the sensor-mounted region 20A is inserted into the living body in a direction indicated by the arrow Y1. An end R1 shown in FIG. 15 is a part of the sensor-mounted region 20A that faces the extraction region 20B. When the end R1 has a rounded shape, the sensor-mounted region 20A is easily inserted into the living body.

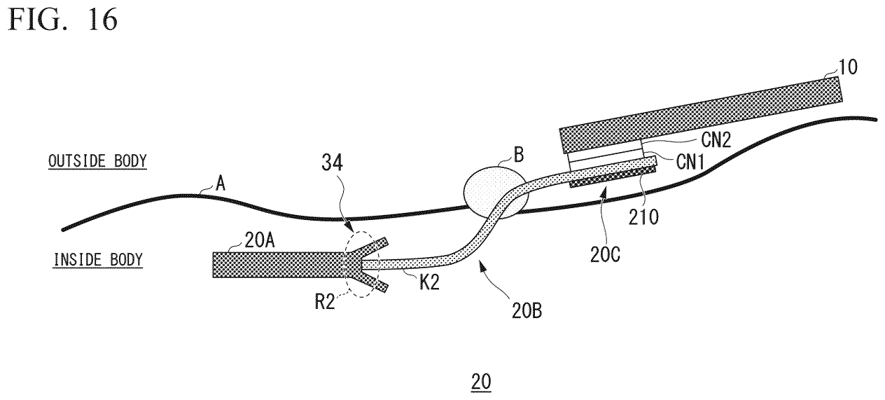

In addition, the sensor-mounted region 20A may have a fall-off prevention structure on the side of the extraction region 20B to prevent the sensor-mounted region 20A from falling off of the living body. An anchor 34 shown in FIG. 16 is an example of the fall-off prevention structure. The anchor 34 is provided at an end R2 which is a part of the sensor-mounted region 20A that is on the side of the extraction region 20B.

The anchor 34 is provided, for example, at an end of the sensor unit substrate 20 which is on the side of the control unit substrate 10. The anchor 34 is provided at the end in a direction in which it spreads in a direction opposite to a direction in which the sensor-mounted region 20A is inserted from the end into the living body. Here, the anchor 34 may be provided at a part of the sensor unit substrate 20 closer to the center than the end of the control unit substrate 10.

Even if a force is applied to the extraction region 20B, the connector region 20C, or the control unit substrate 10 from the outside toward the outside of the living body, the sensor-mounted region 20A is less likely to fall off of the living body due to the fall-off prevention structure.

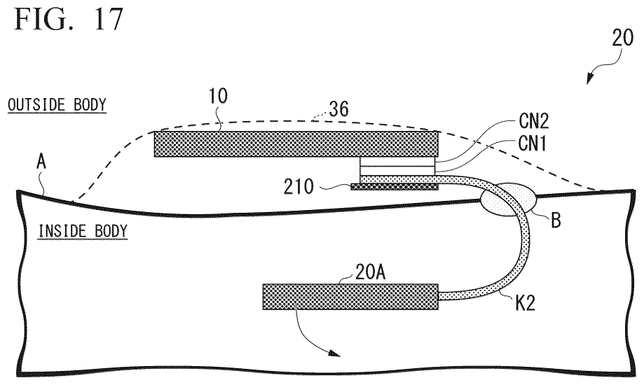

In addition, the sensor unit substrate 20 may have a pressure applying mechanism for pressing the sensor-mounted region 20A against the inside of the living body. In the example shown in FIG. 17, the extraction region 20B of the sensor unit substrate 20 is formed of a rigid-flexible substrate 35, and the control unit substrate 10 is fixed to the skin A by a fixing unit 36. The fixing unit 36 is, for example, an adhesive tape such as a surgical tape. The fixing unit 36 is attached to the surface opposite to the skin A of the control unit substrate 10 so that the fixing unit 36 covers the control unit substrate 10, and thus the control unit substrate 10 is fixed to the skin A. The rigid-flexible substrate 35 and the fixing unit 36 are an example of the pressure applying mechanism. The sensor-mounted region 20A is biased toward the living body due to the repulsive force of the rigid-flexible substrate 35. By being biased by the rigid-flexible substrate 35, the sensor-mounted region 20A can be prevented from being lifted up.

Consequently, the sensor 20X mounted in the sensor-mounted region 20A can be stably held at a desired measurement position, and thus stable measurement results can be obtained.

Here, the method by which the fixing unit 36 fixes the control unit substrate 10 is not limited to the example shown in FIG. 17. As another example, a double-sided tape may be used as the fixing unit 36, the fixing unit 36 may be attached to the surface of the control unit substrate 10 that faces the skin A, and thus the control unit substrate 10 may be fixed to the skin A. In addition, the fixing unit 36 may be a member other than an adhesive tape such as a band.

In addition, when the control unit substrate 10 is sufficiently heavy, the fixing unit 36 may be omitted from the pressure applying mechanism. For example, if it is expected that there will be little movement of a part of the living body to which the biological monitoring device 1 is attached while the biological monitoring device 1 operates, the control unit substrate 10 may be fixed to the skin A due to its own weight. In this case, the control unit substrate 10 has a weight large enough to prevent it from being lifted up from the skin A due to the repulsive force of the rigid-flexible substrate 35 and has a weight small enough not to apply an excessive pressure to the skin A.

As described above, according to the present embodiment, an effect of reducing the burden on the living body in which the sensor is implanted is obtained.

While forms for implementing the present invention have been described above with reference to embodiments, the present invention is not limited to the embodiments at all, and various modifications and substitutions can be made without departing from the spirit and scope of the present invention. The configurations described in the above embodiments and the examples may be combined.

1. A biological monitoring device, comprising:
a sensor unit substrate on which one or more sensors are mounted; and
a control unit substrate on which a power source, a control unit and a storage unit are mounted,
wherein the sensor unit substrate has a sensor-mounted region in which the sensor is mounted and an extraction region having a power source line and a signal line between it and the control unit substrate, and the sensor-mounted region is implanted in a living body.

2. (canceled)

3. The biological monitoring device according to claim 1, wherein the sensor unit substrate has a displacement prevention unit that prevents the position of the sensor unit substrate in the sensor-mounted region from being shifted, and the displacement prevention unit has a protrusion.

4. The biological monitoring device according to claim 2, wherein the sensor unit substrate has a displacement prevention unit that prevents the position of the sensor unit substrate in the sensor-mounted region from being shifted, and the displacement prevention unit has a hole.

5. (canceled)

6. The biological monitoring device according to claim 1, wherein the sensor unit substrate has a displacement prevention unit that prevents the position of the sensor unit substrate in the sensor-mounted region from being shifted, and
the displacement prevention unit is a part where at least a part of the surface of the sensor unit substrate has been surface-treated with a chemical substance that promotes adhesion to biological tissue.

7. The biological monitoring device according to claim 1, wherein the sensor unit substrate has a displacement prevention unit that prevents the position of the sensor unit substrate in the sensor-mounted region from being shifted, and the displacement prevention unit is a fine unevenness provided on at least a part of the surface of the sensor unit substrate.

8. (canceled)

9. (canceled)

10. (canceled)

11. (canceled)

12. (canceled)

13. The biological monitoring device according to claim 1, wherein the sensor-mounted region has a fall-off prevention structure on the side of the extraction region to prevent the sensor-mounted region from falling off of the living body.

14. The biological monitoring device according to claim 1, wherein the sensor-mounted region has a rounded shape on the side opposite to the extraction region.

15. (canceled)

16. The biological monitoring device according to claim 1, wherein, in the sensor unit substrate, the sensor is mounted on the surface that is inside the body when the sensor is implanted in the living body between both surfaces of the sensor-mounted region.

17. (canceled)

18. The biological monitoring device according to claim 1, wherein, in the sensor unit substrate, ends of the sensor-mounted region are chamfered.

19. (canceled)

20. The biological monitoring device according to claim 1, further comprising a clock unit,
wherein on the control unit substrate, a wireless communication unit is additionally mounted,
the storage unit stores the sensor output signal in association with a recording time, which is a time measured by the clock unit,
the control unit causes the wireless communication unit to wirelessly transmit a predetermined signal to an external device at a predetermined transmission period based on the time measured by the clock unit,
the control unit causes the wireless communication unit to wirelessly transmit the sensor output signal stored in the storage unit and the recording time associated with the sensor output signal to the external device, and
the external device corrects time series data of the sensor output signal and the recording time wirelessly received by the external device based on a reception time of the predetermined signal wirelessly received by the external device.

21. (canceled)

22. The biological monitoring device according to any one of claim 1, wherein the control unit notifies a user of whether the installation position of the sensor is normal.

23. The biological monitoring device according to claim 1, wherein the sensor-mounted region and the control unit substrate are non-flexible, and the extraction region is flexible.

24. The biological monitoring device according to claim 1, wherein, in the sensor unit substrate, the extraction region is disposed across the inside of the living body and the outside of the living body, and the control unit substrate is disposed outside the living body.

25. The biological monitoring device according to claim 1, wherein the extraction region has a constricted shape.

26. (canceled)

27. (canceled)

28. (canceled)

29. The biological monitoring device according to claim 1, wherein the extraction region has a meander shape formed by a plurality of curved parts.

30. The biological monitoring device according to claim 1, wherein the extraction region has a wedge shape.

31. The biological monitoring device according to claim 1, wherein the sensor unit substrate has a pressure applying mechanism for pressing the sensor-mounted region against the inside of the living body.

32. (canceled)

33. (canceled)

34. (canceled)

35. (canceled)

36. (canceled)
